(19) United States
(12) Patent Application Publication
(10) Pub. No.: US 2021/0023280 A1
(43) Pub. Date: Jan. 28, 2021

(54) Medical Lavage Device

(71) Applicants: Anthony G. Stanley, North Bay Village, FL (US); STANLEY MEDICAL DEVICES, INC., North Bay Village, FL (US)

(72) Inventors: Anthony G. Stanley, North Bay Village, FL (US); George Moss Stanley, North Bay Village, FL (US)

(21) Appl. No.: 16/935,986

(22) Filed: Jul. 22, 2020

Related U.S. Application Data
(60) Provisional application No. 62/877,400, filed on Jul. 23, 2019.

(51) Int. Cl. A61M 1/00

(52) U.S. Cl. CPC A61M 1/0064; A61M 1/0009; A61M 2205/587; A61M 2210/0662; A61M 2205/36; A61M 2205/8206; A61M 2210/0612; A61M 2205/273

(57) ABSTRACT

Medical lavage devices are described. Devices include a container that can retain a lavage liquid and an outlet through which a lavage liquid carried by the container can be delivered. Devices also include a light source that is retained relative to the outlet such that the light (e.g., white light, UV light) from the light source can illuminate a liquid delivery area, i.e., an area through which liquid will pass or be delivered following exit from the container via the outlet. A device can optionally include a pump for moving liquid through the outlet and/or an attachment at the outlet, e.g., a luer lock, that can be used to attach an accessory, e.g., a delivery catheter to the device. A device can include one or more accessory components, e.g., a splash guard, a magnifying lens, a penetration depth guard, etc., to improve or modify the liquid delivery characteristics of the device.

### CROSS REFERENCE TO RELATED APPLICATION

This application claims filing benefit of U.S. Provisional Patent Application Ser. No. 62/877,400, having a filing date Jul. 23, 2019, which is incorporated herein by reference in its entirety.

### BACKGROUND

Lavage is one of the most common medical treatments, and it encompasses the therapeutic washing of both internal and external organs or body parts, including gastric lavage, eye lavage, ear lavage, and wound lavage, just to name a few. For instance, ear lavage for removal of ear wax or foreign matter is carried out an estimated 150,000 times each week in the US alone.

Proper lavage protocols require both visualization of the area to be treated and irrigation of the area with a lavage fluid of choice; it generally includes multiple repetitions to ensure complete cleaning. The multi-step method can introduce complications and expense. For instance, when considering an ear lavage protocol, as illustrated in FIG. 1 and FIG. 2, a first step requires examination of the ear canal 10 by use of an otoscope 12 to identify the location of the material to be removed (FIG. 1). Following, a medical practitioner can then use an irrigation device 14 to deliver the lavage fluid 16 into the ear canal 10 at the proper location to contact and remove the extraneous material 18 without damage or excessive irritation to the canal wall or the ear drum. Unfortunately, every individual will vary somewhat with regard to, e.g., shape, direction, and length of the ear canal, as well as location of adjacent nerves (e.g., the auricular branch of the facial nerve and the auricular branch of the vagus nerve) and other structures that could be damaged or irritated by the protocol. As such, the lavage protocol can require patience, dexterity, and in many cases, assistance for positioning of the ear and manipulation of the ear canal so as to alternately visualize the area and irrigate the area. For instance, it is generally necessary to manipulate the outer ear with one hand to modify the shape/location of the ear canal while alternately handling either the irrigation device 14 (FIG. 2) or the otoscope 12 (FIG. 1). Similar issues exist in other lavage protocols in which the practitioner is attempting to both see and irrigate a small and/or difficult to visualize area.

What is needed in the art is a medical device that can provide for simultaneous viewing and lavage of an area.

### SUMMARY

According to one embodiment, disclosed is a medical lavage device that includes a container that can retain a lavage liquid and an outlet through which a lavage liquid carried by the container can be delivered. The device also includes a light source that is retained and delivered relative to the outlet such that the light source can illuminate a liquid delivery area, i.e., an area through which liquid will pass or be delivered following exit from the container via the outlet. A device can optionally include a pump for moving liquid through the outlet and/or an attachment at the outlet, e.g., a luer lock, that can be used to attach an accessory, e.g., a delivery catheter, a splash guard, a magnifying lens, a penetration guard, etc., to improve or modify one or more characteristics of the device.

Also disclosed are methods for utilization of a medical lavage device. For instance, a method can include an ear lavage protocol, an eye lavage protocol, or a wound lavage protocol. A method can include simultaneously illuminating, visualizing, and irrigating an area by use of the device. In one embodiment, a method can also include simultaneously manipulating an area of a subject's body (e.g., the ear canal, the eyelid, a wound area, etc.), while also illuminating, visualizing, and irrigating the area, with all activities being carried out by a single medical practitioner.

### BRIEF DESCRIPTION OF THE FIGURES

A full and enabling disclosure of the present subject matter, including the best mode thereof to one of ordinary skill in the art, is set forth more particularly in the remainder of the specification, including reference to the accompanying figures in which:

FIG. 1 illustrates examination of an ear canal by use of an otoscope according to known practice.

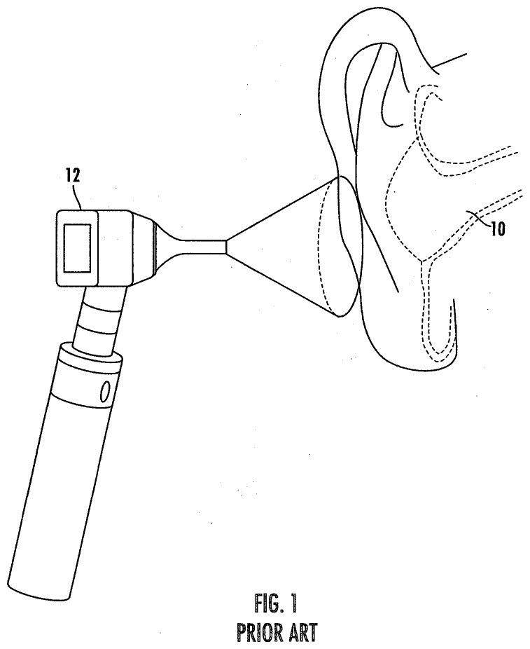

FIG. 2 illustrates a typical prior art ear canal irrigation protocol.

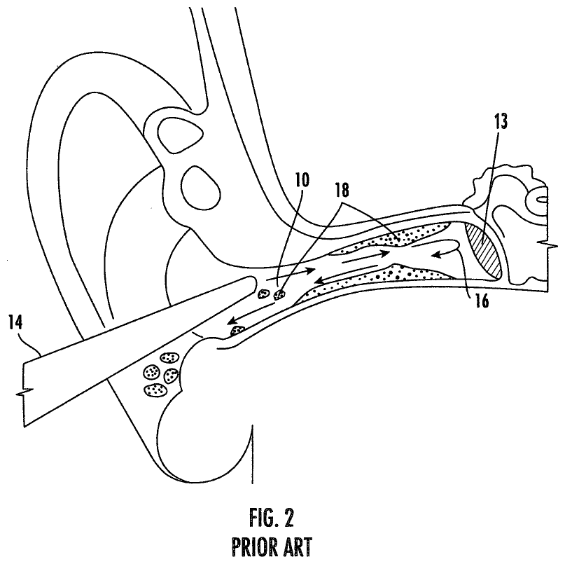

FIG. 3 illustrates one embodiment of a medical lavage device described herein.

FIG. 4 illustrates another embodiment of a medical lavage device described herein.

FIG. 5 illustrates another embodiment of a medical lavage device described herein.

FIG. 6 illustrates another embodiment of a medical lavage device described herein.

FIG. 7 illustrates another embodiment of a medical lavage device described herein.

FIG. 8 illustrates components that can be included in a kit that includes a medical lavage device as described herein, optionally in conjunction with one or more additional components.

Repeat use of reference characters in the present specification and drawings is intended to represent the same or analogous features or elements of the present invention.

### DETAILED DESCRIPTION

Reference will now be made in detail to various embodiments of the disclosed subject matter, one or more examples of which are set forth below. Each embodiment is provided by way of explanation of the subject matter, not limitation thereof. In fact, it will be apparent to those skilled in the art that various modifications and variations may be made in the present disclosure without departing from the scope or spirit of the subject matter. For instance, features illustrated or described as part of one embodiment, may be used in another embodiment to yield a still further embodiment.

Medical lavage devices are disclosed that can provide for simultaneous illumination, visualization, and irrigation of an area in need of lavage. Also disclosed are methods for utilizing the devices. The medical lavage devices can be utilized to simultaneously illuminate and irrigate an area by a single practitioner and, as such, better visualize an area during a lavage protocol. Moreover, a practitioner can simultaneously illuminate and irrigate an area and still have a free hand for other activities. For instance, a practitioner can have a free hand for manipulation of the area to be irrigated or can use the free hand for carrying another device, e.g., a suture needle, probe, or the like. Disclosed devices can provide benefit to the art, such as by preventing accidental trauma and irritation to a patient as has been previously known to happen due to an inability to visualize an area clearly during irrigation. Disclosed devices can also save time and money, as the single operator use of the device can remove the necessity for a second practitioner to be present during a lavage protocol.

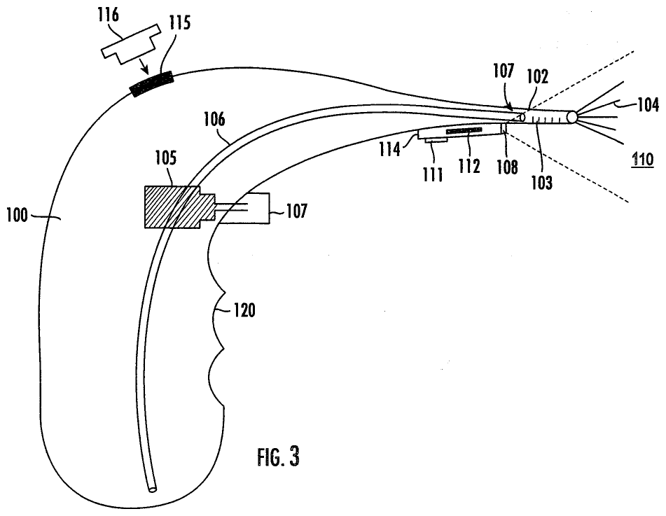

FIG. 3 illustrates one embodiment of a medical lavage device. As illustrated, a device can include a container 100 that can be of a size to hold an amount of a lavage liquid (e.g., water, a saline solution, a fluorescein solution, or a water-based cleansing solution) expected to be used in a protocol. For instance, when considering an ear, eye, or wound lavage protocol, a container 100 can generally be designed to carry up to about 750 milliliters (mL) of a lavage liquid; for instance, from about 100 mL to about 600 mL, or from about 300 mL to about 500 mL, in some embodiments.

The container 100 can be formed of any suitable medical grade material. In general, the container 100 can be formed of a polymeric material (e.g., a molded polyolefin, polyvinyl chloride, polystyrene, or the like) that can be either disposable for single use applications or can be reusable and optionally sterilizable.

The container can include an outlet 102 through which lavage liquid can be delivered to an irrigation area 104. In some embodiments, the outlet 102 can be adjustable so as to modify flow delivery through the outlet 102. For instance, the shape of the outlet 102 can be varied (e.g., increase and decrease of an aperture size at the outlet) so as to modify the flow exiting the outlet from a narrow stream spray to a wide cone spray or a wide and flat fan spray. In one embodiment, the outlet 102 an include a rotatable nozzle that can rotate about an axis to place one or more of multiple different orifices in fluid communication with a delivery line 106. The different orifices can have different shapes and sizes so as to modify the shape and/or pressure of a fluid stream as it exits the outlet 102.

In one embodiment, the outlet 102 can include an attachment 107 by which an accessory device (also referred to herein as a component) can be secured to the device. By way of example, a delivery catheter 103 can be secured to a device by use of attachment 107 at the outlet 102. Any suitable attachment 107 can be included, such as, and without limitation to, a luer taper fitting (also generally referred to as a luer lock), a slip tip fitting, a quick-connect fitting, etc.

Attachment 107 can be sized to secure one or more of multiple different accessory devices. For example, an attachment 107 can secure any of a number of different fluid delivery catheters 103 that can vary with regard to size and shape specific for a particular embodiment. When utilized, a fluid delivery catheter 103 can generally vary with regard to both length and gauge depending upon the size of the patient (e.g., child, adult, etc.) and the particular lavage protocol. For example, when considering an ear lavage protocol, it may be preferred to utilize a relatively long fluid delivery catheter 103 as compared to an eye lavage procedure. When carrying out an ear lavage, a fluid delivery catheter can also be helpful in holding aside the tragus while delivering the fluid to the ear canal, which can also improve visualization of the area. In an eye lavage protocol, a shorter delivery catheter may be preferred. The gauge of a deliver catheter 103 can be of any size but can generally be up to about 20 gauge, e.g., from about 16 gauge to about 20 gauge. A delivery catheter 103 can generally be disposable and can be used with a container 100 that is either disposable or reusable.

An attachment 107 is not limited to use with delivery catheters and can be utilized to attach other components of a system, alternatively or in addition to a delivery catheter 103. For instance, and as discussed further herein, an attachment 107 can be utilized to attach a splash guard, a magnifying lens, a penetration guard, or any other useful attachment to a device.

Referring again to FIG. 3, at or near the outlet 102, a device can include a light source 108. The light source 108 can be located such that a light emitted from the light source 108 can illuminate an area that includes an irrigation area 104. The light source can be any suitable type, and in one embodiment, can include at least one light emitting diode (LED). The device can also include related electronics necessary for the light source 108 to emit a light from the general area of the outlet 102 in an irrigation area 104. In general, the light source 108 can be designed to emit white light, but this is not a requirement, and in other embodiments, light of another color may be preferred.

In one embodiment, a light source 108 can deliver ultra-violet (UV) light to an irrigation area 104. UV light can be desirable in examination procedures in which a luminescent material is visualized. For instance, bioluminescent pathogens including bacterial and fungal pathogens can be visually detected by use of UV light. Disorders such as tinea capitis, pityriasis versicolor, vitiligo, and melasma, among others, can be detected by inclusion of a UV-emitting light source 108 on a device. Diagnostic materials can also be detected by UV light. For instance, fluorescein solution can be applied to an area (e.g., the surface of an eye). Upon examination under UV light, corneal abrasions or scratches can then be visually detectable. Following examination, the fluorescein can be rinsed out by use of the lavage protocol. In such an embodiment, a container 100 can carry the fluorescein solution for initial application or optionally, the water solution for rinsing the fluorescein following visualization. In one embodiment, a device can carry two containers, one container that holds a detection solution, e.g., a fluorescein solution, and a second container that holds a lavage solution. A switch can be used to control which solution is delivered to an irrigation area 104. In another embodiment, two devices can be sequentially utilized, the first carrying a detection solution and the second carrying a lavage solution.

The light source 108 can be secured to the device in any fashion. For instance, in the illustrated embodiment of FIG. 3, the light source 108 is at a surface of the device near the outlet 102. In other embodiments, a light source can be within a wall of the device (e.g., formed into the wall) or held within a cut-out of a wall of the device. A light source 108 can be secured to a device by use of an attachment mechanism (e.g., a screw, bolt, strap, etc.) or an adhesive or can be held within a housing 114 that can be welded or bonded, e.g., melt bonded, adhesively bonded, etc. to a wall of the container or any other portion of the device.

In one embodiment, the light source 108 can be removable and replaceable. For instance, the light source 108 can be designed for replacement when a light emission device, e.g., a diode, becomes inoperable. In one embodiment, a device can allow replacement of a first light source, e.g., a white light emitting device, with a second light source, e.g., a UV emitting light. Thus, a device can be utilized for both visual inspection in standard conditions, e.g., with a white light, and for visual inspection using UV light, e.g., to visually detect a luminescent pathogen or detection solution. In yet another embodiment, the light source 108 can carry two different light emission devices, e.g., both a white LED emission source and a UV source. In this embodiment, the device can include electrical components, wires, switches, etc., examples of which are discussed further herein, that can allow the user to select which light emission is operating. Thus, during use the light emission in the irrigation area 104 can be modified as desired.

A housing 114 can carry the light source 108 (e.g., a white LED, UV source, multiple light emitting devices, etc.), as well as one or more electrical components, e.g., wires, switch 111, power source 112, necessary for proper function of the light source 108. A switch 111 can be in electrical communication with the light source 108 and can be a single-use switch or a multi-use switch. For instance, in those embodiments in which a device is a single-use, disposable device, the switch 111 can be a single-use switch, e.g., a tab formed of a non-conductive material. Upon removal of the tab, a circuit is closed and the light source 108 will be powered to emit light in the irrigation site 110. However, any switch type may alternatively be utilized including, without limitation, a slide switch, a toggle switch, a button switch, etc. For instance, a switch 111 can be a simple on/off switch to supply power to a single light source or can include the capability to switch between different light emitting devices.

The light source 108 can be in electrical connection with a power supply 112, such as a battery 112. A battery 112 can be within a housing 114 that carries the light source 108 and the switch 111, as shown in FIG. 3, or can be located elsewhere on a device as desired.

The size and type of the power supply can vary as is known, generally depending upon the load expected and whether the device is intended to be a single-use or a multi-use device. For instance, a device can include one or more 1.5V button-type batteries 112 to power the light source 108 in some embodiments.

A device can also include a pump 105 within the container 100 to pump a liquid from the container body 100 to the outlet 102 through a delivery line 106. A pump 105 can be manual or powered, as desired. For instance, in the embodiment illustrated in FIG. 3, a manual pump 105 is included in the device that is powered by the operator activating the pump via a trigger 107. A manual pump 105 can function according to standard practice, in which the pump 105 includes internal check valves (not shown) and liquid is drawn into the pump 105 from the container 100 and then forced out of the outlet 102 when the trigger 107 is activated.

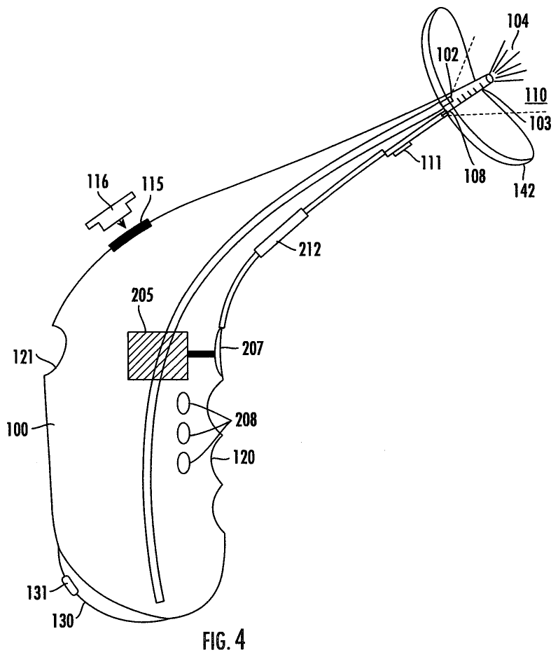

In an alternative embodiment, illustrated in FIG. 4, a device can include an automatic, powered pump 205. In this embodiment, the pump 205 can be in electrical communication with a switch 207 and a power supply 212, e.g., one or more batteries, as discussed previously. Optionally, the power supply 212 can also provide power for the light source 108 via the light switch 111, though the two components can have separate power supplies in some embodiments.

A pump, either manual or automatic, can include adjustment capabilities so as to adjust the flow of fluid from the outlet 102 during use. Adjustments can include those that affect, without limitation, pressure of fluid at the outlet, flow rate through the outlet, continuous or pulsatile flow through the outlet, etc. For instance, when considering an automatic pump 205, the pump can include a timer that can be set to deliver a pulsed stream from the outlet, as well as to deliver the pulsed flow with a predetermined rate of pulsing for the flow. In addition, an automatic pump 205 can include adjustments that can modify the power, and hence, the rate of fluid delivery and pressure of fluid delivery from the outlet 102 to the irrigation area 104.

A pump 205 can include electronics and switches as are known in the art to provide for the desired flow control. For instance, a pump 205 can include one or more switches 208 or the like on a surface of the container 100 by which the operating parameters for the pump 205 can be set. Optionally, an automatic pump and power supply can be detachable and reusable in those embodiments in which the remainder of the device is a single-use, disposable device.

A device can include a port 115 and mated cap 116 by which the container 100 can be filled with the desired lavage fluid. The cap can be designed for multiple use devices (e.g., removable and resealable), as well as for single-use devices (e.g., destroyed upon initial use), as desired.

A device can optionally include an ergonomically designed handle 120 that can include one or more finger grips, as shown, and optionally, with a thumb grip 121 (FIG. 4) for improved gripping of a device during use. Moreover, the surface of a device can be varied to improve grip. For instance, the finger/thumb grip areas of a device can include a non-slip surface for improved gripping, in some embodiments.

The overall shape of a device is not particularly limited and can vary depending upon the type(s) of lavage that are intended to be carried out by use of the devices. For instance, in the embodiment of FIG. 3, the overall shape of the device can be such that the outlet 102 is at an angle of approximate 90° to the finger grips of the handle 120. It may be beneficial in other embodiments to vary this angle. For instance, in the embodiment of FIG. 4, the angle between the handle 120 and the outlet 102 is greater, approximately, 120°.

As illustrated in FIG. 4, in one embodiment, a device can include a heater 130, e.g., a resistive heater, that can be utilized to heat a liquid held in the container 100 to a desired temperature for an irrigation protocol. For instance, a heater 130 can heat a lavage liquid held in a container 100 to a temperature of from about 80° F. to about 100° F., or from about 85° F. to about 95° F. in some embodiments. A heater 130 can include a switch 131 that can be used to turn the heater on upon filling the container 100 with water. A switch 131 can be any suitable switch, examples of which include those described previously for use with the light source 108. For instance, a switch 131 can be a one-time switch for use with a disposable device or can be intended for use multiple times in a reusable device. A heater 130 can also include a power source that can power only the heater 130 or that can be in electrical communication with several components of a device and can power the heater 130, as well as, e.g., a pump 205 and a light source 108.

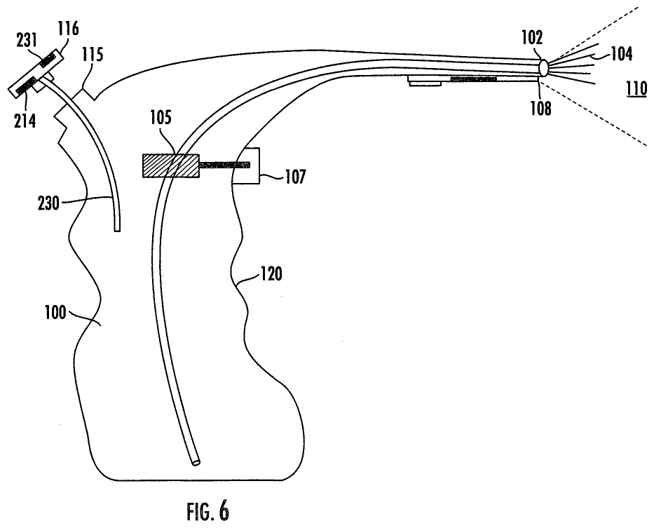

As illustrated in FIG. 4, a heater 130 can be within the container 100, for instance at or near a wall of the container 100. In another embodiment, a heater can be removable from the container or located in a different area of a container. For instance, FIG. 6 illustrates another embodiment of a device that includes a heater 230 that is attached to a cap 116. As shown, the heater can be controlled by use of a switch 231 and powered by a battery 214 that can be retained within or on the cap 116 in this embodiment. The device of FIG. 6 can include a light source 108, pump 105, etc. as discussed previously, with variations as previously described. For instance, in this particular embodiment, the device is illustrated with no accessory device at the outlet 102 and shown with the fluid delivered directly from the outlet 102 to the irrigation area 104 that is within the illuminated area 110. The embodiment of FIG. 6 also has a slightly different shape as compared to other illustrated embodiments. The overall shape of a device is not particularly limited, and a device can be designed both for ergonomics, as well as proper visualization and delivery of fluid to an irrigation area, as will be understood by one of ordinary skill in the art.

A device can include the capability for attachment of components that can be useful in a protocol. For instance, as illustrated in FIG. 4, a device can include a penetration depth shield 142 that can be removably attached to the outlet 102 or a delivery catheter 103. In one embodiment, a penetration depth shield 142 can be simply slid over the outlet end of a device to a desired distance, and can be capable of use with other devices. For instance, a penetration depth shield can also be utilized with a typical ear bulb syringe as is known in the art and commonly used for clearing ear or nose canals of small children. A penetration depth shield 142 can be formed of a transparent material, so as to avoid interfering with the visualization of the area; for instance, a clear plastic that can be slid over the end of the outlet 102 to a desired depth.

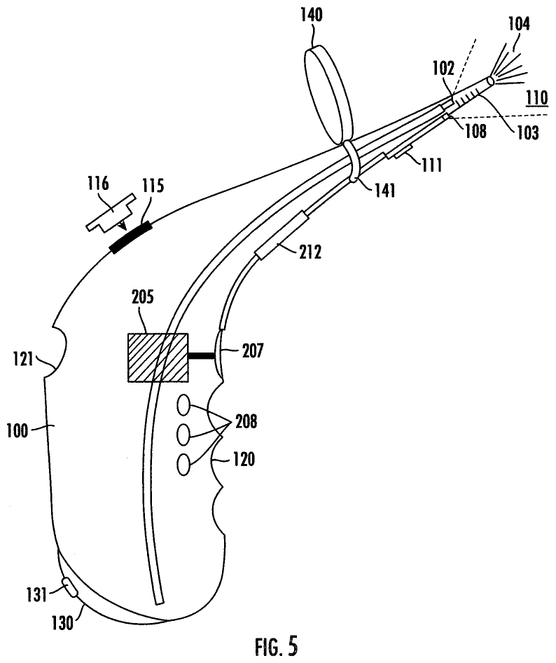

As indicated in FIG. 4 and FIG. 5, the outlet 102 and/or delivery catheter 103 can optionally include distance markings, which can be utilized to determine penetration depth of the outlet 102 into a patient, e.g., an ear canal or a wound, so as to prevent excessive penetration depth. Distance markings on a device can generally be in millimeters.

FIG. 5 illustrates one embodiment of a device that includes a magnifying lens 140 held on the device by use of a clamp 141 as another example of an attachment that can be included with a device. A magnifying lens 140 can be clamped to a device so as to be located between the irrigation area 104 and the user so as to enable improved view of the irrigation area. Any sort of a clamp 141 can be used to retain a magnifying lens 140. In one embodiment, the clamp 141 can allow a range of motion of the magnifying lens 140 in order that a user can place the lens of the glass 140 at the most convenient location for viewing.

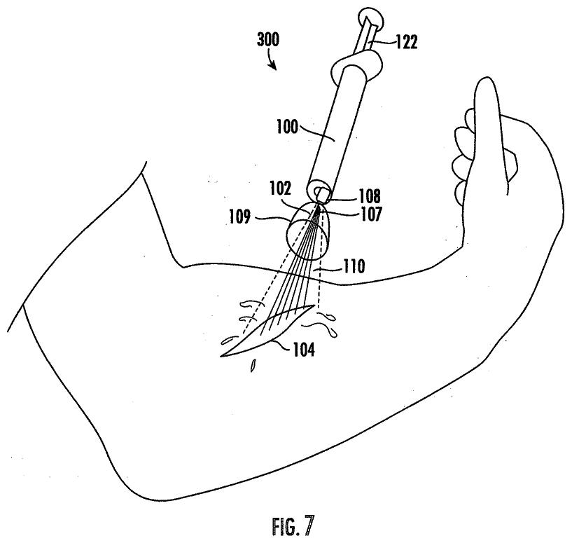

FIG. 7 illustrates another embodiment of a medical lavage device 300. As illustrated, in this embodiment, a device can be aligned on a single axis, with no angle along the length of the device from the outlet 102 to the opposite end of the device, which in this embodiment is the end of a plunger 122. As with other embodiments, the device 300 includes a light source 108 near the outlet 102. In addition, the device includes a container 100 for carrying a lavage fluid. The light source 108 is located such that it can illuminate an area 110 that encompasses irrigation area 104 which, in this embodiment, includes a wound. As shown, the attachment 107 of device 300 has been secured to a splash guard 109, as another example of a useful attachment that can be utilized in conjunction with a lavage device as described herein.

In addition, device 300 utilizes a plunger 122 to expel fluid from the container 100 out of the outlet 102. However, any sort of fluid motive system, e.g., a manual or automatic pump as described previously, or a single plunger as illustrated in FIG. 6 or any other suitable motive system, can be utilized with a container of any desired shape or size, as would be evident to one of skill in the art.

A medical lavage device as described can be provided in one embodiment as a kit that can include the device itself in conjunction with one or more additional components, i.e., components that can be utilized together or separately. For instance, in one embodiment, a kit can include a medical lavage device in conjunction with an attachment that can be unitary or separable from the device, and the attachment can be utilized to connect other components to the medical lavage device. Other components can include, without limitation, one or more fluid delivery catheters of various lengths, gauges, etc.; one or more light sources that can be removably locatable on the device (e.g., alternative white, UV, IR, or colored bulbs or LEDs); additional batteries and/or a battery charger or charging source (e.g., a 120V charging station); a separable heating/cooling station to retain the device between uses; a connectable pump; multiple caps for the container (e.g., a heating cap, as well as a non-heating cap); a penetration depth shield; a magnifying lens; a splash guard; etc.

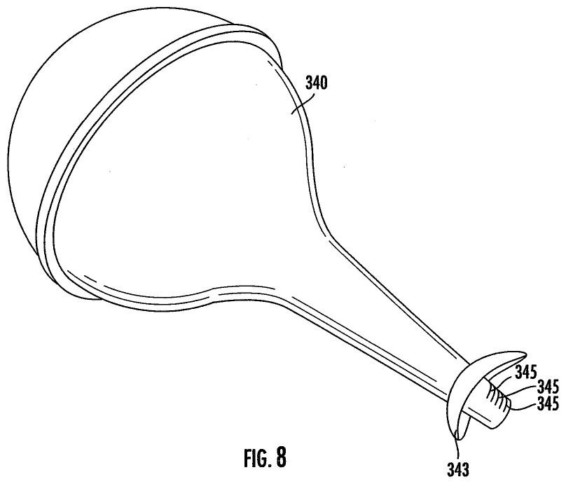

FIG. 8 illustrates one embodiment of components that can be included in a kit that also includes the medical lavage device. As illustrated, a system (or kit) can include a bulb syringe 340 as is typically utilized for lavage or suctioning small areas, generally the ear, nose, or mouth of a small child. In this embodiment, a system can include both a bulb syringe 340 that includes distance markings 345 at the insertion end of the device as well as a removable penetration depth shield 343. The distance markings 345 and the penetration depth shield 343 can prevent insertion of the end of the bulb syringe 340 to a potentially dangerous depth. A kit can also include a medical lavage device as discussed previously, and the penetration depth shield 343 can be removably attached to either the medical lavage device (e.g., as illustrated in FIG. 4) or to the bulb syringe 340.

While certain embodiments of the disclosed subject matter have been described using specific terms, such description is for illustrative purposes only, and it is to be understood that changes and variations may be made without departing from the spirit or scope of the subject matter.

What is claimed is:

1. A medical lavage device comprising:
a container;
a liquid outlet in fluid communication with the container;
a light source retained relative to the liquid outlet such that the light source illuminates a liquid delivery area for liquid delivered from the container through the outlet.

2. The device of claim 1, further comprising a pump in fluid communication with the container and the outlet.

3. The device of claim 2, further comprising a power source in electrical communication with the pump.

4. The device of claim 3, wherein the power source is in electrical communication with the light source.

5. The device of claim 1, further comprising an attachment.

6. The device of claim 5, the attachment comprising a luer taper fitting.

7. The device of claim 1, the device comprising distance markings at the liquid outlet.

8. The device of claim 1, the light source comprising a light emitting diode and/or an ultra-violet light emitting device.

9. The device of claim 1, wherein the container defines a volume of about 600 milliliters or less.

10. The device of claim 1, wherein the devise is a single-use, disposable device.

11. The device of claim 1, further comprising a heater configured to heat a liquid held within the container.

12. A kit comprising the medical lavage device of claim 1 and one or more accessory devices.

13. The kit of claim 12, the accessory devices comprising one or more delivery catheters, a magnifying lens, a splash guard, a penetration depth guard, a bulb syringe, one or more batteries, one or more light sources, or a combination thereof.

14. The kit of claim 13, the accessory devices comprising multiple delivery catheters that differ from one another with regard to length and/or gauge.

15. The kit of claim 13, the one or more accessory devices comprising a bulb syringe and a penetration depth guard, the penetration depth guard being removable attachable to both the bulb syringe and the medical lavage device.

16. A method for irrigating an area in a medical treatment, the method comprising:
directing a light at the area, the light being emitted from a light source of a device, the device comprising a container;
delivering a liquid from the container to the area in conjunction with the directing of the light at the area.

17. The method of claim 16, further comprising manipulating the area, wherein the manipulation is carried out by the operator of the device of claim 1.

18. The method of claim 16, wherein the method is an ear lavage method, an eye lavage method, or a wound lavage method.

19. The method of claim 16, wherein the light comprises white light or ultra-violet light.

20. The method of claim 16, wherein the liquid comprises a detection solution, the method further comprising delivering a second liquid to the area, the second liquid comprising a rinsing solution.
